(19) United States
(12) Patent Application Publication
(10) Pub. No.: US 2026/0053877 A1
(43) Pub. Date: Feb. 26, 2026

(54) COMPOSITION FOR ALLEVIATING, PREVENTING OR TREATING KIDNEY DISEASE OR VASCULAR DISEASE COMPRISING PANAX GINSENG EXTRACTS FERMENTED BY LACTOBACILLUS PLANTARUM, AND PREPARATION METHOD THEREOF

(71) Applicant: DAMEUM Inc., Jeonbuk-do (KR)

(72) Inventors: So Chong KIM, Jeonbuk-do (KR); Dong Yoon SHIN, Jeonbuk-do (KR)

(21) Appl. No.: 19/306,238

(22) Filed: Aug. 21, 2025

(30) Foreign Application Priority Data
Aug. 22, 2024 (KR) 10-2024-0112509

(51) Int. Cl. A61K 36/258; A61K 35/747; A61P 3/06

(52) U.S. Cl. CPC A61K 36/258; A61K 35/747; A61P 3/06; A61K 2236/19

(57) ABSTRACT

The present invention relates to a composition for alleviating, preventing, or treating a kidney disease or a vascular disease accompanied by hypoxia, the composition comprising a Panax ginseng extract fermented with a Lactobacillus plantarum strain. When the Lactobacillus palantarum strain is treated with cells, hypoxia-suppressing and cholesterol accumulation-lowering effects are exhibited. Therefore, the composition can be effectively used in a composition for alleviating, preventing, or treating a kidney disease or a vascular disease accompanied by hypoxia.

### CROSS REFERENCE TO RELATED APPLICATION

This application claims priority to Korean Patent Application No. 10-2024-0112509, filed on Aug. 22, 2024. The entire disclosure of the application identified in this paragraph is incorporated herein by reference.

### SEQUENCE LISTING

This application contains references to amino acid sequences and/or nucleic acid sequences which have been submitted concurrently herewith as the sequence listing XML file entitled “000405us_SequenceListing.XML”, file size 8,126 bytes bytes, created on 21 Aug. 2025. The aforementioned sequence listing is hereby incorporated by reference in its entirety pursuant to 37 C.F.R. § 1.52(e)(5).

### FIELD

The present invention relates to a composition for alleviating, preventing or treating a kidney disease or vascular disease accompanied by hypoxia, comprising a Panax ginseng extract fermented with a Lactobacillus plantarum strain, and preparation method thereof.

### BACKGROUND

With the development of medical technology and science, adult diseases and metabolic diseases such as kidney disease, diabetes, and dementia are increasing in modern people whose lifespan has been extended. In addition, infectious diseases are increasing due to environmental pollution and warming, and patients suffering from these diseases have a poor quality of life due to diseases.

As interest in health promotion has increased since the recent outbreak of coronavirus (COVID-19), modern people prefer natural-derived pollution-free health foods. As a result, interest in natural plants that have effects such as boosting immunity, antioxidant activity, and promoting metabolism, have fewer side effects in the human body and help promote health has increased. As the physiological activity of natural plants is scientifically verified, the demand for healthy foods is increasing, and research and industry of related products are developing.

Panax ginseng, one of many natural plants that promote and support human health, grows in mountainous regions between 30° and 48° north latitude and is found naturally in Korea and China, and has light yellowish brown or grayish brown roots, and has vertical and thin root marks. The head part of root is slightly bent, and the rhizome has a stem residue attached, has a unique smell, and the taste is sweet and bitter. Ginseng is known to be effective in inhibiting cancer cell proliferation, rejuvenating, enhancing immunity, strengthening digestive function, and strengthening metabolism.

Ginsenoside, one of the ingredients found in ginseng, is a type of saponin and a glycoside extracted from ginseng, and accounts for approximately 5.2% of ginseng. The ginsenoside contained in ginseng, and polyphenol which is an antioxidant active ingredient, are known to have effects such as lowering blood sugar, promoting the secretion of glucocorticoids, which are adrenocortical hormones, suppressing the proliferation of cancer cells, removing toxic oxygen, strengthening immunity against various diseases, antipyretic action, promoting development, lowering blood pressure, promoting respiration, suppressing the rise of blood sugar, increasing the number of red blood cells and hemoglobin, and enhancing the digestive tract.

Natural raw materials that are harmless to the human body and beneficial to health contain large amounts of polymer materials. Such a polymer material is known to not accurately exhibit efficacy due to its low absorption rate into the human body. Fermentation is the decomposition of organic compounds by microorganisms such as yeast and bacteria, and the high molecular substance contained in natural raw materials can be converted into low molecular substance that is easy to absorb into the human body by using fermentation.

Lactobacillus plantarum is a bacterium that acts on the fermentation of soy sauce and soybean paste, has about 5.5% resistance to salt, and can ferment arabinose, glucose, fructose, galactose, maltose, sucrose, dextran, raffinose, trehalose, and the like.

### SUMMARY

### Technical Problem

Accordingly, the present inventors measured the anti-hypoxia and cholesterol accumulation-lowering activities of Panax ginseng extract fermented with a Lactobacillus plantarum strain, and completed the present invention for a composition for alleviating, preventing, or treating kidney disease or vascular disease accompanied by hypoxia.

Accordingly, an aspect of the present disclosure is to provide a A health functional food composition for alleviating a kidney disease or vascular disease accompanied by hypoxia, comprising a Panax ginseng extract fermented with a Lactobacillus plantarum strain.

Another aspect of the present disclosure is to provide a pharmaceutical composition for preventing or treating a kidney disease or vascular disease accompanied by hypoxia, comprising a Panax ginseng extract fermented with a Lactobacillus plantarum strain.

Another aspect of the present disclosure is to provide a method for preparing a health functional food composition for alleviating a kidney disease or a vascular disease accompanied by hypoxia: a fermentation step of inoculating a strain of Lactobacillus plantarum into a Panax ginseng extract and fermenting the same.

### Solution to Problem

The present invention relates to a composition for alleviating, preventing or treating a kidney disease or vascular disease accompanied by hypoxia, containing a Panax ginseng extract fermented with a Lactobacillus plantarum strain, and a method for preparing the same.

Hereinafter, a detailed description will begiven of the present disclosure.

One aspect of the present disclosure relates to a health functional food composition for alleviating a kidney disease or vascular disease accompanied by hypoxia, comprising a Panax ginseng extract fermented with a Lactobacillus plantarum strain.

In an embodiment of the present disclosure, the kidney disease accompanied by hypoxia may be selected from the group consisting of kidney cancer, kidney infarction, chronic renal failure, renal artery stenosis, and glomerulonephritis, but is not limited thereto.

In an embodiment of the present disclosure, the vascular disease may be selected from the group consisting of arteriosclerosis, non-alcoholic fatty liver, and hyperlipidemia, but is not limited thereto.

The ginseng extract may contain various types of ginsenosides. Specifically, for example, the ginseng extract may contain Rg1, Rb1, Rg3s, Rk1, Rg5, Rd, and the like. In general, Rg1 is effective in improving learning function and acting as antipyretics, Rb1 is effective in suppressing central nervous system, antipyretic, analgesic, and protecting liver function, and Rg3s is effective in suppressing cancer cell metastasis, lowering blood pressure, and suppressing anticancer drug resistance. In addition, it has been reported that Rg5 has effects such as inhibitory effects on neuroinflammation, pulmonary inflammation, and anti-inflammatory effects on skin inflammatory reactions, and Rd has effects such as promoting secretion of adrenocortical hormones.

When the health functional food composition of the present disclosure is used as a food additive, the health functional food composition may be added as it is or used together with other foods or food ingredients, and may be appropriately used according to a conventional method. In general, the health functional food composition of the present invention may be added in an amount of 15% by weight or less, preferably 10% by weight or less, based on the raw material when preparing a food or beverage.

There is no particular limitation on the type of the food. Examples of the food to which the substance may be added include meat, sausages, bread, chocolate, candy, snacks, confectionery, pizza, ramen, other noodles, gums, dairy products including ice cream, various soups, beverages, tea, drinks, alcoholic beverages, health functional foods, and vitamin composites, and the like, and all foods in a common sense are included.

The health functional food may be selected from the group consisting of capsules, tablets, powders, granules, liquid, pills, syrups, and bars.

The beverage may contain various flavoring agents or natural carbohydrates as additional ingredients. The natural carbohydrates may be monosaccharides such as glucose and fructose, disaccharides such as maltose and sucrose, natural sweeteners such as dextrin and cyclodextrin, synthetic sweeteners such as saccharin and aspartame, and the like. The ratio of the natural carbohydrate may be appropriately determined by the selection of those skilled in the art.

In addition to the above, the health functional food composition of the present disclosure may contain various nutrients, vitamins, electrolytes, flavoring agents, colorants, pectic acid and salts thereof, alginic acid and salts thereof, organic acids, protective colloidal thickeners, pH adjusting agents, stabilizers, preservatives, glycerin, alcohols, carbonating agents used in carbonated beverages, and the like. In addition, the health functional food composition of the present invention may contain flesh for the preparation of natural fruit juice, fruit juice beverages, and vegetable beverages. These components may be used independently or in combination. The ratio of such additives can also be appropriately selected by those skilled in the art.

Another aspect of the present disclosure relates to a pharmaceutical composition for preventing or treating a kidney disease or a vascular disease accompanied by hypoxia comprising a Panax ginseng extract fermented with a Lactobacillus plantarum strain.

In an embodiment of the present disclosure, the kidney disease accompanied by hypoxia may be selected from the group consisting of kidney cancer, kidney infarction, chronic renal failure, renal artery stenosis, and glomerulonephritis, but is not limited thereto.

In an embodiment of the present disclosure, the vascular disease may be selected from the group consisting of arteriosclerosis, non-alcoholic fatty liver, and hyperlipidemia, but is not limited thereto.

The pharmaceutical composition of the present disclosure may be prepared in a unit dose form by being formulated using a pharmaceutically acceptable carrier and/or excipient, or may be prepared by being inserted into a multi-dose container, according to a method that can be easily carried out by a person having ordinary skill in the art to which the present invention belongs. In this case, the formulation may be in the form of a solution, a suspension, or an emulsion in an oil or aqueous medium, or in the form of an concentrated Extract, a powder, a granule, a tablet, a capsule or a gel (e.g., a hydrogel), and may further include a dispersing agent or a stabilizing agent.

Another aspect of the present disclosure relates to a method for preparing a health functional food composition for alleviating a kidney disease or a vascular disease accompanied by hypoxia: a fermentation step of inoculating a strain of Lactobacillus plantarum into a Panax ginseng extract and fermenting the same.

In an embodiment of the present disclosure, the kidney disease accompanied by hypoxia may be selected from the group consisting of kidney cancer, kidney infarction, chronic renal failure, renal artery stenosis, and glomerulonephritis, but is not limited thereto.

In an embodiment of the present disclosure, the vascular disease may be selected from the group consisting of arteriosclerosis, non-alcoholic fatty liver, and hyperlipidemia, but is not limited thereto.

In an embodiment of the present disclosure, the fermentation step may be performed at 20 to 50° C., preferably at 20 to 45° C., 20 to 40° C., 25 to 50° C., 25 to 45° C., 25 to 40° C., 30 to 50° C., or 30 to 45° C., for example, at 30 to 40° C., but is not limited thereto.

In an embodiment of the present disclosure, the fermentation step may be performed for 3 to 10 days, preferably 3 to 7 days, 3 to 5 days, 4 to 10 days, 4 to 7 days, or 4 to 5 days, for example, may be performed for 5 days, but is not limited thereto.

### Advantageous Effects of Invention

The present disclosure relates to a composition for alleviating, preventing, or treating a kidney disease or a vascular disease accompanied by hypoxia, the composition comprising a Panax ginseng extract fermented with a Lactobacillus plantarum strain. When the Lactobacillus palantarum strain is treated with cells, hypoxia-suppressing and cholesterol accumulation-lowering effects are exhibited. Therefore, the composition can be effectively used in a composition for alleviating, preventing, or treating a kidney disease or a vascular disease accompanied by hypoxia.

### BRIEF DESCRIPTION OF DRAWINGS

FIG. 1 is a graph showing cytotoxicity of a Panax ginseng extract fermented with a Lactobacillus plantarum strain according to an embodiment of the present disclosure.

FIG. 2 is a graph showing the effect of inhibiting cell death due to hypoxia of a Panax ginseng extract fermented with a Lactobacillus plantarum strain according to an embodiment of the present disclosure.

FIG. 3A is a graph showing the measurement of the activity of HMG-CoA reductase following treatment with a Panax ginseng extract fermented with a Lactobacillus plantarum strain according to an embodiment of the present disclosure.

FIG. 3B is a graph showing the measurement of the activity of LDLR according to the treatment of Panax ginseng extract fermented with Lactobacillus plantarum strain according to an embodiment of the present disclosure.

FIG. 3C is a graph showing the measurement of the activity of SREBP2 according to the treatment of Panax ginseng extract fermented with Lactobacillus plantarum strain according to an embodiment of the present disclosure.

### DETAILED DESCRIPTION

Hereinafter, a better understanding of the present disclosure may be obtained through the following examples, which are set forth to illustrate, but are not to be construed to limit, the scope of the present disclosure.

Throughout this specification, the “%” used to indicate the concentration of a particular substance is, unless otherwise stated, (weight/weight)% of solids/solids, (weight/volume)% of solids/liquids, and (volume/volume)% of liquids/liquids.

### Experimental Example 1. Preparation of Panax Ginseng Extract Powder Fermented with Lactobacillus Plantarum Strain

Panax ginseng was finely chopped and dried, and 2.5 liters of water was added to the pulverized ginseng, and reflux extraction was performed three times for 4 hours to prepare Panax ginseng extract powder. Water was then added 10 times to the prepared Panax ginseng extract powder, and Lactobacillus plantarum strains were added in an amount of 5% based on the total weight and fermented at 30-40° C. for 120 hours. The fermentation liquid was completely dried using a freeze dryer, then prepared as a powder, and named KG227, and stored at −20° C. for use in the experiment.

### Experimental Example 2. Cell Culture

Human hepatocarcinoma, HK-2 cells were cultured in DMEM (Gibco, Carlsbad, CA, USA) supplemented with 10% FBS (GW Vitek, Seoul, South Korea) and 1% penicillin/streptomycin (Welgene, Gyeongsang, South Korea) in a 75T flask at 37° C. under 5% CO₂ using a CO₂ incubator (SMA-30D; ASTEC Korea, Seoul, South Korea).

### Experimental Example 3. Measurement of mRNA Expression Using qPCR

Total RNA was extracted using a GeneALL hybrid-R 100p kit (GeneAll, Korea) to extract intracellular mRNA, and RNA concentration was measured using a NanoDrop spectrophotometer. cDNA synthesis was performed using a Perfect Real Time (PrimeScipt RT Master Mix). The intracellular mRNA expression level was performed using qPCR, PCR was performed using TB green Premix Ex Taq, and the nucleotide sequences of the primers for PCR are shown in Table 1 below. Quantitative analysis, relative to each gene, from the PCR results was calculated as the relative ratio of PCR products to GAPDH.

TABLE 1

| SEQ | Sequence | |
|---|---|---|
| ID NO: | Name | Sequence(5′→3′) |
| 1 | GAPDH-F | CTGCCCCCTCTGCTGATG |
| 2 | GAPDH-R | AGGAGGCARTGCTGATGATCTT |
| 3 | SREBP2-R | TGAATGACCGTTGCACTGAAG |
| 4 | SREBP2-F | CCTGGGAGACATCGACGAGAT |
| 5 | LDLR-F | TCTGCAACATGGCTAGAGACT |
| 6 | LDLR-R | TCCAAGCATTCGTTGGTCCC |
| 7 | HMGCR-F | TGATTGACCTTTCCAGAGCAAG |
| 8 | HMGCR-R | CTAAAATTGCCATTCCACGAGC |

### Experimental Example 4. Statistical Processing

For statistical processing of all experiments, the mean±standard deviation was calculated using Sigma plot (window version 10.0, USA) program.

### Example 1. Measurement of Cytotoxicity of KG227

The cytotoxicity of the KG227 test solution prepared in Experimental Example 1 was evaluated by the MTT assay method using proximal tubular blood cells (HK-2). First, HK-2 cells distributed from the Korean Cell Line Bank were cultured in 24 well plate for 24 hours, and then the KG227 test solution was treated at concentrations of 10 μg/mL and 20 μg/mL, respectively, and cultured for 24 hours. MTT (Sigma-Aldrich, ST. 0.5 mg/mL of Louis, MO, USA) was treated for 2 hours, and after all media were removed, crystals were dissolved with 200 μL DMSO. Thereafter, the absorbance was measured at 570 nm using a spectrophotometer (VersaMax™) to compare cell viability.

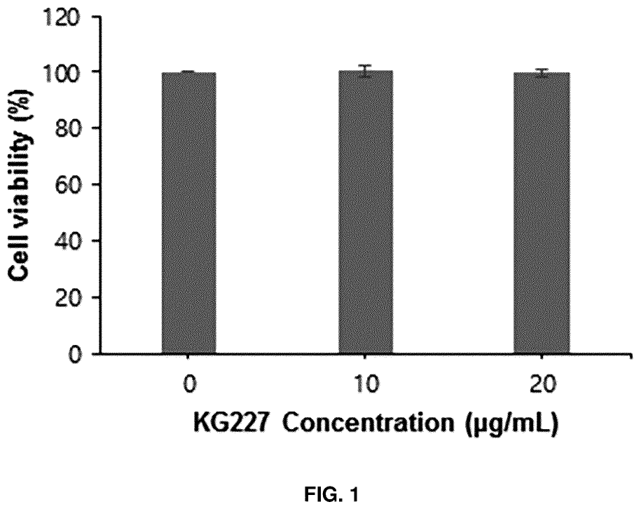

As shown in FIG. 1, since the KG227 test solution did not show toxicity at concentrations of 10 μg/mL and 20 μg/mL, it was determined that it would not affect future cell experiments, and the activity was measured using the corresponding concentration.

### Example 2. Measurement of Hypoxia-Induced Cell Death Inhibitory Effect of KG227

HK-2 cells were incubated in a culture medium under conditions of 1% O₂, 5% CO₂, 94% N₂, and 37° C. for 24 hours to induce hypoxia. In order to measure the effect of KG227 on inhibition of cell death by hypoxia, the KG227 test solution at the time of hypoxia induction was treated with 0 μg/mL, 10 μg/mL, and 20 μg/mL, respectively, and the cell viability was compared and shown in Table 2.

TABLE 2

| | Cell Viability |
|---|---|
| Negative control (NC) | 72.4 ± 3.2% |
| 10 μg/mL Treatment Group | 89.6 ± 2.3% |
| 20 μg/mL Treatment Group | 84.1 ± 1.2% |

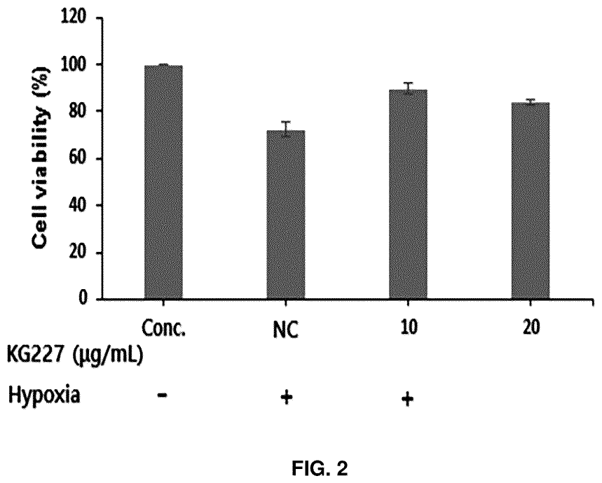

As shown in FIG. 2, the cell viability of hypoxia-induced cells was measured as 72.4±3.2% in the negative control group, 89.6±2.3% in the 10 μg/mL treatment group of the KG227 test solution, and 84.1±1.2% in the 20 μg/mL treatment group.

When hypoxia occurs due to the pressure of cells caused by kidney diseases such as kidney cancer, the secretion of erythropoietin, which is a red blood cell hematopoietic hormone, may increase, thereby increasing the number of red blood cells, and thus may cause headache and dizziness. Therefore, it can be inferred that the inhibitory effect of KG227 on cell death by hypoxia is effective in relieving kidney disease.

### Example 3. Activity Measurement of HMG-CoA Reductase, LDLR, and SREBP2 According to KG227 Treatment

First, the effect of KG227 on the activity of HMG-CoA (hydroxy-methylglutaryl-coenzyme A) reductase involved in cholesterol synthesis pathway was analyzed.

Specifically, HepG2 cells cultured for 24 hours were treated with a KG227 test solution at 10 μg/mL and 20 μg/mL for 2 hours, respectively, and then washed twice with cold phosphate-buffered saline (PBS) by treating with 100 μg/mL LO (linear acid-oleic acid complex) for 24 hours, and then the cells were collected, mRNA was extracted, and the activity of HMG-COA reductase was measured with qPCR and shown in Table 3.

TABLE 3

| | Fold change |
|---|---|
| Controls (LO Conc.) | 1.69 ± 0.03 |
| 10 μg/mL Treatment Group | 1.61 ± 0.09 |
| 20 μg/mL Treatment Group | 1.41 ± 0.14 |

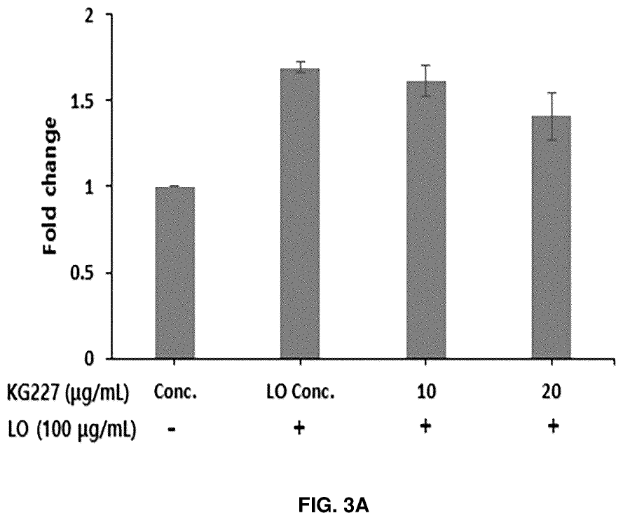

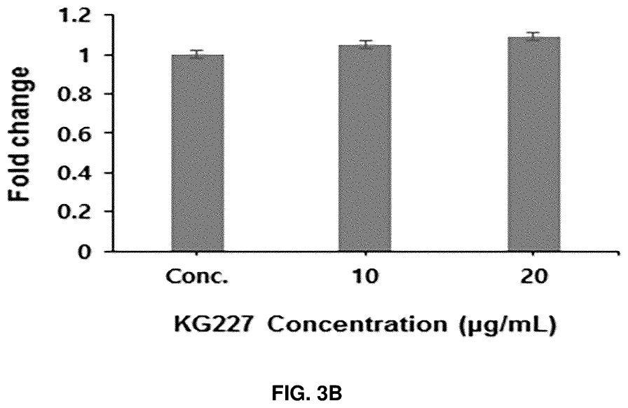

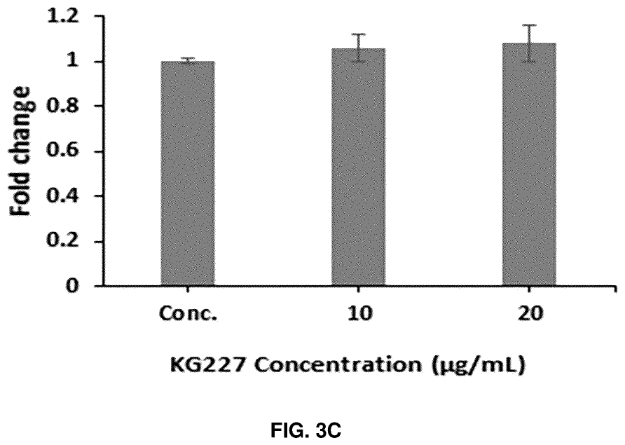

As shown in FIG. 3A, the group that induced lipid accumulation with LO increased the activity of HMG-CoA reductase to 1.69±0.03. This appears to be an increase in the activity of HMG-CoA reductase with increased lipid accumulation or with increased dietary fat intake.

In the case of the treatment group treated with the KG227 test solution, the HMG-CoA reductase activity was 1.61±0.09 in the 10 μg/mL treatment group and 1.41±0.14 in the 20 μg/mL treatment group, which was lower than that of the untreated group, and thus it was confirmed that the KG227 test solution had an effect of inhibiting HMG-CoA activity.

Then, the effect of KG227 on the activity of LDLR (low-density lipoprotein receptor) and SREBP2 (sterol regulatory element-binding protein2) was analyzed.

Specifically, the KG227 test solution was treated with 10 μg/mL and 20 μg/mL in HepG2 cells and then incubated for 24 hours. The control group was evaluated for efficacy by substituting serum free media instead of the KG227 test solution, and the results are shown in Table 4.

TABLE 4

Fold change

| | LDLR | SREBP2 |
|---|---|---|
| 10 μg/mL Treatment Group | 1.05 ± 0.02 | 1.06 ± 0.06 |
| 20 μg/mL Treatment Group | 1.09 ± 0.02 | 1.08 ± 0.07 |

As shown in FIG. 3B, the LDLR activity in HepG2 cells was measured as 1.05±0.02 in the KG227 test solution 10 μg/mL-treated group and 1.09±0.02 in the 20 μg/mL-treated group. Furthermore, the SREBP2 activity, as shown in FIG. 3C, was measured as 1.06±0.06 in the KG227 test solution 10 μg/mL-treated group and 1.08±0.07 in the 20 μg/mL-treated group.

LDLR is present in the cell membrane, binds to low-density lipoprotein/cholesterol, and serves to absorb into the cell, releasing cholesterol available for inhibition of HMG-CoA reductase activity. In addition, SREBP2 is a transcription factor that regulates cholesterol synthesis and LDLR expression, and it has been reported that increased expression of LDLR and SREBP2 generally positively affects cholesterol metabolism.

Cholesterol is a lipid in cell membranes that protects the body's cell membrane and blood vessel wall and prolongs the life of red blood cells. Insufficient cholesterol in the body shortens the lifespan of red blood cells, which can lead to anemia and hemorrhagic diseases, but high blood cholesterol can lead to the formation of sediments in the walls of blood vessels, causing arteriosclerosis.

According to the above Example, it can be confirmed that the activity of HMG-CoA reductase is suppressed and the expression of LDLR and SREBP2 factors is increased according to the KG227 treatment, and therefore, it can be inferred that lipid and cholesterol accumulation is reduced, thereby helping blood circulation and being effective for vascular diseases.

What is claimed is:

1. A health functional food composition for alleviating a kidney disease or vascular disease accompanied by hypoxia, comprising a Panax ginseng extract fermented with a Lactobacillus plantarum strain.

2. The health functional food composition of claim 1, wherein the kidney disease accompanied by hypoxia is selected from the group consisting of kidney cancer, kidney infarction, chronic renal failure, renal artery stenosis, and glomerulonephritis.

3. The health functional food composition of claim 1, wherein the vascular disease is selected from the group consisting of arteriosclerosis, non-alcoholic fatty liver, and hyperlipidemia.

4. A pharmaceutical composition for preventing or treating a kidney disease or vascular disease accompanied by hypoxia, comprising a Panax ginseng extract fermented with a Lactobacillus plantarum strain.

5. The pharmaceutical composition of claim 4, wherein the kidney disease accompanied by hypoxia is selected from the group consisting of kidney cancer, kidney infarction, chronic renal failure, renal artery stenosis, and glomerulonephritis.

6. The pharmaceutical composition of claim 4, wherein the vascular disease is selected from the group consisting of arteriosclerosis, non-alcoholic fatty liver, and hyperlipidemia.

7. A method for preparing a health functional food composition for alleviating a kidney disease or a vascular disease accompanied by hypoxia:
a fermentation step of inoculating a strain of Lactobacillus plantarum into a Panax ginseng extract and fermenting the same.

8. The method for preparing a health functional food composition according to claim 7, wherein the kidney disease accompanied by hypoxia is selected from the group consisting of kidney cancer, kidney infarction, chronic renal failure, renal artery stenosis, and glomerulonephritis.

9. The method of claim 7, wherein the vascular disease is selected from the group consisting of arteriosclerosis, non-alcoholic fatty liver, and hyperlipidemia.

10. The method for preparing a health functional food composition according to claim 7, wherein the fermentation step is performed at 20 to 50° C.

11. The method for preparing a health functional food composition of claim 7, wherein the fermentation step is performed for 3 to 10 days.


SEQUENCE LISTING (8 sequences)

SEQ ID NO: 1 - RNA, 18 nt, Homo sp.
  source 1..18: mol_type = other RNA
ctgccccctctgctgatg

SEQ ID NO: 2 - RNA, 22 nt, Homo sp.
  source 1..22: mol_type = other RNA
aggaggcartgctgatgatctt

SEQ ID NO: 3 - RNA, 21 nt, Homo sp.
  source 1..21: mol_type = other RNA
tgaatgaccgttgcactgaag

SEQ ID NO: 4 - RNA, 21 nt, Homo sp.
  source 1..21: mol_type = other RNA
cctgggagacatcgacgagat

SEQ ID NO: 5 - RNA, 21 nt, Homo sp.
  source 1..21: mol_type = other RNA
tctgcaacatggctagagact

SEQ ID NO: 6 - RNA, 20 nt, Homo sp.
  source 1..20: mol_type = other RNA
tccaagcattcgttggtccc

SEQ ID NO: 7 - RNA, 22 nt, Homo sp.
  source 1..22: mol_type = other RNA
tgattgacctttccagagcaag

SEQ ID NO: 8 - RNA, 22 nt, Homo sp.
  source 1..22: mol_type = other RNA
ctaaaattgccattccacgagc